(19) United States
(12) Patent Application Publication
(10) Pub. No.: US 2021/0308398 A1
(43) Pub. Date: Oct. 7, 2021

(54) NITROUS OXIDE SEDATION ADMINISTRATION SYSTEM

(71) Applicant: Gary Porter Associates LLC, Warrington, PA (US)

(72) Inventor: Gary K PORTER, Warrington, PA (US)

(21) Appl. No.: 17/277,208

(22) PCT Filed: Sep. 18, 2019
(86) PCT No.: PCT/US2019/051742

§ 371 Date: Mar. 17, 2021

Related U.S. Application Data
(60) Provisional application No. 62/732,790, filed on Sep. 18, 2018.

(51) Int. Cl. A61M 16/01; A61M 16/00; A61M 16/20

(52) U.S. Cl. CPC A61M 16/01; A61M 16/0078; A61M 16/06; A61M 2202/0468; A61M 2016/003; A61M 16/208

(57) ABSTRACT

Described herein are methods and systems for delivering an analgesic to a patient. The systems and methods including a breathing bag that has an outer shell, which creates a hollow inter cavity. The breathing bag also has an opening at first end, and an opening secured by a check valve at a second end. A tube, or set of tubes, may then be attached to the breathing bag, such that at least one aperture along a portion of the tube is housed within the breathing bag. The check-valve of the breathing bag having an inlet, an outlet, an air passage, a tube attachment, and a flapper. Gas can then pass through the apertures, into the breathing bag, and out to the patient via the check valve.

### TECHNICAL FIELD

This present disclosure relates generally to a system for administering gaseous anesthesia to a patient.

### BACKGROUND

Certain procedures (e.g., invasive dental work) may require the administration of analgesia to a patient in order to either block pain that is incurred during the procedure or make the experience more pleasant for the patient. Often, this analgesia takes the form of a local anesthesia such as lidocaine that is administered to the patient through a syringe and needle arrangement and is injected at the site, such as the gum area, where the procedure is to be performed. Lidocaine is typically administered prior to the procedure.

In addition to this local anesthetic, many procedures involve the administration of nitrous oxide sedation to the patient. Unlike lidocaine, nitrous oxide is gaseous in form and is administered to the patient before and during the course of the procedure. As such, nitrous oxide is administered not as a general anesthetic but to serve as a mild analgesic and relaxant for the patient.

Generally, in order to administer an analgesic gas to a patient, a nasal mask is used. Examples of such masks are the patient nasal masks manufactured by Porter Instrument Division, Parker Hannifin Although such nasal delivery masks, and in particular the masks manufactured by Porter, perform well and currently constitute the state-of-the-art in nasal masks, some improvements are possible with regard to the functioning of the system.

### BRIEF DESCRIPTION OF THE DRAWINGS

Some embodiments are illustrated by way of example and not limitation in the figures of the accompanying drawings in which:

FIG. 1A is an illustrative example of an existing analgesia system.

FIG. 1B is an illustrative example of a new light-weight analgesia system according to an example embodiment.

FIG. 2 is a diagram illustrating a new light-weight analgesic system including a breathing bag according to an example embodiment.

FIG. 3 is a diagram illustrating a detailed view of a breathing bag according to an example embodiment.

FIG. 4 is a diagram illustrating a detailed view of a check-valve system according to an example embodiment.

FIG. 5A is a diagram illustrating a detailed view of a flapper of a check-valve system according to an example embodiment.

FIG. 5B is a diagram illustrating a detailed view of an air passage component of a check-valve system according to an example embodiment.

FIG. 5C is a diagram illustrating a detailed view of an air passage component of a check-valve system according to another example embodiment.

FIG. 6A is a diagram illustrating a view of a square breathing bag according to an example embodiment.

FIG. 6B is a diagram illustrating a view of a round breathing bag according to an example embodiment.

### DETAILED DESCRIPTION

The present description and claims may make use of the terms “a,” “at least one of,” and “one or more of,” with regard to particular features and elements of the illustrative embodiments. It should be appreciated that these terms and phrases are intended to state that there is at least one of the particular feature or element present in the particular illustrative embodiment, but that more than one can also be present. That is, these terms/phrases are not intended to limit the description or claims to a single feature/element being present or require that a plurality of such features/elements be present. To the contrary, these terms/phrases only require at least a single feature/element with the possibility of a plurality of such features/elements being within the scope of the description and claims.

In addition, it should be appreciated that the following description uses a plurality of examples for various elements of the illustrative embodiments to further illustrate example implementations of the illustrative embodiments and to aid in the understanding of the mechanisms of the illustrative embodiments. These examples are intended to be non-limiting and are not exhaustive of the various possibilities for implementing the mechanisms of the illustrative embodiments. It will be apparent to those of ordinary skill in the art in view of the present description that there are many other alternative implementations for these various elements that may be utilized in addition to, or in replacement of, the example provided herein without departing from the spirit and scope of the present disclosure.

As used herein, the term “includes” means “comprise” for example, a device that includes or comprises A and B contains A and B but can optionally contain C or other components other than A and B. A device that includes or comprises A and B may contain A or B, or A and B, and optionally one or more other components such as C.

In accordance with the present invention, various embodiments of an analgesic delivery device are disclosed for use on a patient having a mouth and a nose having a naris. The delivery device is capable of being coupled to a flowmeter system and a vacuum system. Accordingly, one or more embodiments may have an inspiratory gas input for delivering gas to the patient and an exhaust gas output for delivering gas from a patient. The analgesic device comprises an inspiratory gas line having a machine end and a patient end.

The machine end is capable of being fluidly coupled to the inspiratory gas input of the system, and the patient end is configured for being received within the naris of the patient for delivering inspiratory gas to the naris of the patient. An exhaust port is provided that is capable of being fluidly coupled to the exhaust gas output of the system for allowing gas to pass from the interior of the bag to the exhaust gas output of the vacuum system. The exhaust port and vent are capable of cooperatively exerting a negative pressure on the outside air space adjacent to the face mask for preventing inspiratory gas from entering the outside air space adjacent to the face mask and pulling in gases adjacent to the face mask.

When the first Nitrous Oxide Conscious Sedation Unit was developed in the mid-1960s by Harry Langa, D.D.S., it consisted of two flowmeters with valves, one for oxygen and one for nitrous oxide, a breathing bag, connecting tubing, and a nasal inhaler. Over the years, improvements have been made, and safety features have been added. However, the basic units have remained the same. A few years ago, a single-use breathing system, which consists of breathing tubes from the flowmeter, a nasal mask, and scavenging tubing was introduced. However, this system did not include a breathing bag. Due to the reduced size of the tubes in this system, a conventional breathing bag was infeasible. Accordingly, various embodiments disclosed herein are related to a system for a breathing bag that could be conveniently built into this specific system or similar system.

The average respiratory rate in adults is between 12 and 18 breathing cycles per minute. Moreover, the inhalation and exhalation phase of the cycle are not equal. Specifically, the inspiration phase is slightly longer than the expiration phase. As would be expected, when using a gas-based analgesia system, the flow of gases reverses between inhalation and exhalation cycles of a patient. Accordingly, the flowrate of the gases during the two phases is generally in a sinusoidal curve with fairly high flows achieved during a short period of time. Therefore, any breathing circuit used in a gas-based analgesia system must accommodate inflows and outflows, at a minimum resistance, and reverse direction approximately every 2.5 seconds.

Previous systems (e.g., non-rebreathing systems) used in analgesic administration that were common years ago included a gas inlet, a breathing bag, connecting tubing, a non-rebreathing valve (e.g., likely with two check valves), and an anatomical analgesic mask (e.g., covering the mouth and nose).

During operation, the gas was turned on at a flow rate approximating the patient's minute volume, for example 6 L/min, and the bag would begin to inflate. As the patient inhales, the inhaled gas comes directly from the breathing bag. As the exhalation phase starts, the exhalation valve opens to the local atmosphere, and the inhalation check valve closes allowing the breathing bag to refill. This cycle is repeated during each breathing cycle.

In the above example, if the breathing bag were removed, the 6 L/min flow would go directly to the non-rebreathing valve during both the inspiration phase and the expiration phase. This would make it difficult, if not impossible, to have control over what the patient breathes. Thus, the breathing bag is the necessary interface between the continuous flow from the machine and the in and out breathing cycle of the patient.

However, some more recently designed analgesia systems (e.g., the Silhouette) have a much different design. Although the system disclosed herein is applicable to various analgesic systems, for simplicity purposes, various example embodiments may be described herein as they relate to the Porter Silhouette nasal mask. The Porter Silhouette nasal mask is a single-use patient nasal mask and breathing circuit designed with lightweight tubing and a comfortable and conforming nasal mask.

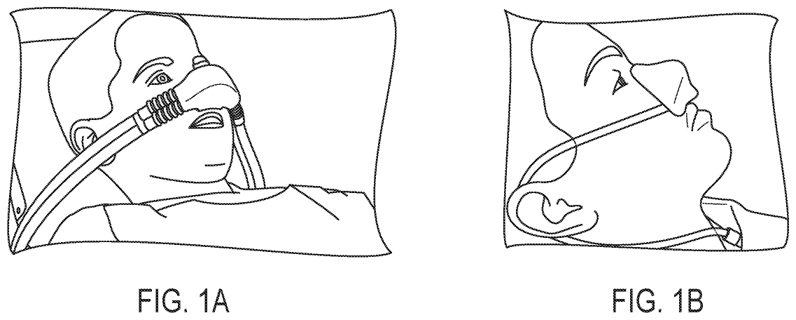

A primary difference between traditional analgesia systems and newer systems (e.g., the Silhouette) is the size of both the mask and tubing. FIG. 1A shows a traditional nasal mask, and FIG. 1B shows an example of a Silhouette nasal mask.

As shown in FIG. 1B, the tubing on the analgesia system has been greatly reduced in diameter. If a normal flow from a flowmeter (e.g., flows of five (5) to six (6) liters per minute) were used and a bag system were placed in a typical location, the bag would quickly fill and stay filled due to the restricted size of the tubing. Another advantage of having a bag system is to monitor the depth and breathing frequency of the patient. However, due to the reduced size of the tubing and system, it would be impossible to use a traditional bag system with the newer analgesia systems.

Thus, current designs allow the gas flow from the flowmeter to go directly to the mask and gas is flowing in both the inspiration and exhalation phase. The velocity of the gas flow allows for plenty of flow during inspiration, and in fact may be more than needed. In the exhalation phase, gas goes directly out the holes in the mask or to the scavenging line.

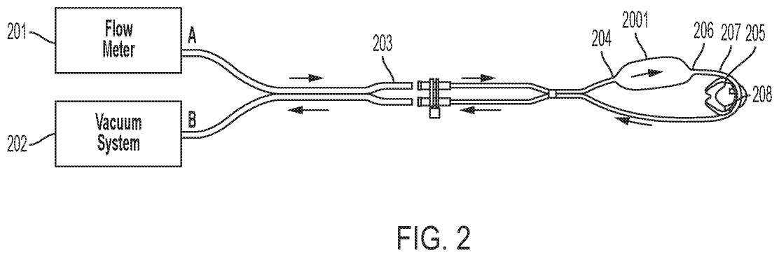

Accordingly, for improved functionality, a breathing bag should be used with the newer lightweight designs. Referring now to FIG. 2, an example system is shown in which a newer light-weight analgesia system (e.g., the Silhouette system) includes a breathing bag according to an embodiment.

In some embodiments, fresh gas flows may be delivered through the outlet of a flowmeter 201 and into the fresh gas tube 203. The fresh gas tube 203 then connects or is inserted 204 into a breathing bag 2001. As discussed further herein, the breathing bag 2001 and the fresh gas tube 203 are bonded to form an air-tight seal.

In further embodiments, and as shown in FIG. 2, the breathing bag 2001 should be located relatively close to the fresh gas inlet of the nasal mask 205. As would be understood by one skilled in the art, the system must not compromise access by a doctor or surgeon (e.g., a dentist) to the patient. However, as should be understood, if the distance between the outlet of the breathing bag 206 and the fresh gas inlet of the nasal mask 205, is large, the inside diameter of the patient adjacent fresh gas tubing 207 must be relative large as well in order to minimize the breathing resistance for the patient. As the patient exhales, gas is pushed out the outlet side of the nasal mask 208. In one or more embodiments, the path of the gas is determined, at least in part, by the check-valve system located inside the breathing bag, which is discussed further herein.

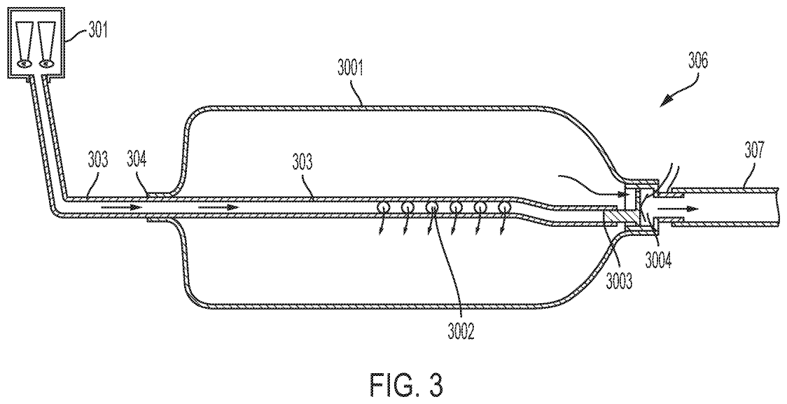

Referring now to FIG. 3, a cross-sectional view of a breathing bag according to various embodiments is shown. The example system of FIG. 3 has a flowmeter 301, a fresh gas tube 303, an air-tight seal 304 between the fresh gas tube and the breathing bag 3001, an outlet 306 of the breathing bag, and a patient-adjacent fresh gas tube 307. It should be understood that the air-tight seal 304 may be created using any known method of adhesion or bonding. In some embodiments, the fresh gas tube 303 traverses the interior of the breathing bag 3001. In a further embodiment, the fresh gas tube 303 may comprise one or more openings 3002. Although the one or more openings 3002 are shown as circular holes or openings, it should be understood that this is only one non-limiting example, and that various other openings types (e.g., slits, spiral cuts, cut outs of any shape, or various mesh tube patterns) are functional within this embodiment.

In some embodiments, the one or more openings 3002 allow for fresh gas to pass into the breathing bag 3001. In addition, the presence of the fresh gas tube 303 provides additional structural integrity to the breathing bag 3001. Specifically, in some embodiments, the fresh gas tube 303 supports the breathing bag 3001 such that it will not collapse due to inadvertent stretching. In another embodiment, the fresh gas tube 303 is attached to a protrusion 3003 within the breathing bag 3001. As further discussed herein, the protrusion 3003 may be of solid construction or hollow. According to some embodiments, the protrusion 3003 may be attached to a check valve system 3004 located at the outlet 306 of the breathing bag 3001.

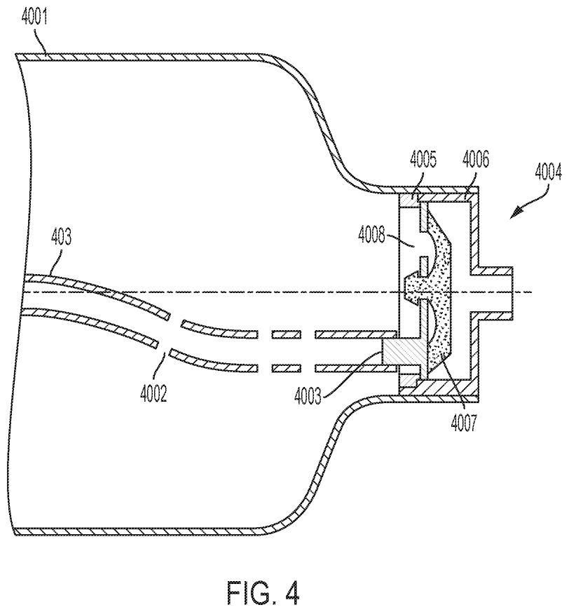

Referring now to FIG. 4, a detailed example embodiment of a check-valve system 4004 is shown. In some embodiments, and as shown, the check-valve system 4004 may comprise an air passage component 4005, a tube attachment component 4006, and a flapper component 4007. According to some embodiments, the air passage component 4005 may comprise one or more openings 4008 to allow fresh gas to pass from the bag 4001 to a patient-adjacent fresh gas tube (not shown). As will be discussed herein, the number and arrangement of the one or more openings 4008 can vary based on need and/or design constraints.

In a further embodiment, the tube attachment component 4006 is securely attached to the air passage component 4005 such that an air-tight seal is formed. As discussed herein with reference to the breathing bag inlet (See FIGS. 2 and 3 at 204 and 304, respectively), this air-tight connection may be formed using any form of adhesive or bonding. In an additional or alternative embodiment, the tube attachment component 4006 and the air passage component 4005 may be singularly constructed (i.e., have a uniform body), such as, for example, molded plastic.

In some embodiments, during the inhalation phase of the breathing cycle, the flapper component 4007 opens (e.g., shifts, flexes, slides, etc.) allowing fresh gas, which may be delivered using the fresh gas tube 403 via the one or more openings 4002, to leave the breathing bag 4001 and flow to the patient-adjacent fresh gas tube (not shown). Alternatively, in some embodiments, the flapper component 4007 closes during the exhalation phase, thus preventing any exhaled gases from entering the breathing bag 4001. As discussed herein, the exhaled gases will then follow the path of least resistance and exit the system via the outlet side of the nasal mask (See FIG. 2 at 208). The exhaled gases are then removed from the patient mask to a vacuum system (See FIG. 2 at 202). In an additional embodiment, during exhalation, while the flapper component 4007 is closed, fresh gases from the flowmeter (See FIG. 2 at 201) will refill the breathing bag 4001 via the one or more openings 4002 in the fresh gas tube 403.

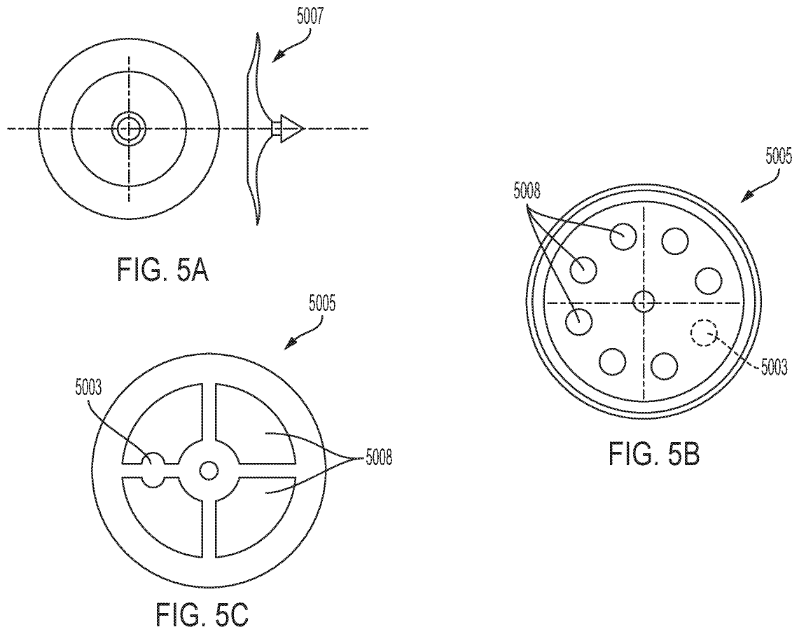

Turning now to FIGS. 5A, 5B, and 5C, detailed example embodiments of the flapper component 5007 and the various designs for the air passage component 5005 are shown. As discussed herein, in some embodiments, the air passage component 5005 may have one or more openings 5008 to allow for the passage of fresh gas to the patient. It should be understood that the number of openings as shown (i.e., 7 in FIG. 5B) is a non-limiting example embodiment, and that some embodiments may have as few as 1 opening, or as many as 1000 micro openings.

Moreover, the protrusion 5003 may be solid in construction or hollow. In an embodiment where the protrusion 5003 is solid, fresh gas may only pass to the patient after passing through the one or more openings (See FIG. 4 at 4002) of the fresh gas tube (See FIG. 4 at 403). In an alternative embodiment, the protrusion 5003 maybe hollow and fresh gas may be passed to the patient by only passing through the fresh gas tube (See FIG. 4 at 403).

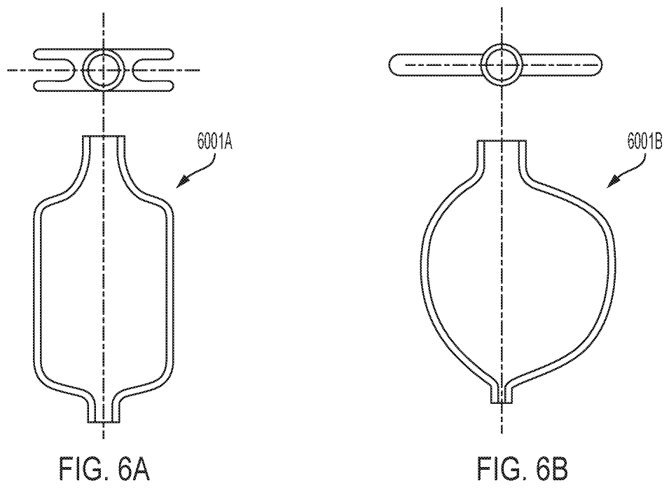

It should be understood that the breathing bag (e.g., 2001, 3001, and 4001) may be of various shapes, as well as sizes. Referring now to FIGS. 6A and 6B, two alternative example designs are shown. FIG. 6A shows an illustrative embodiment of a square bag 6001A, which has sides that collapse inward as the bag deflates. Alternatively, FIG. 6B shows an illustrative embodiment of a round bag 6001B, in which the outer walls extend away from the center when deflated. Various other embodiments may also be possible, such as, for example, a tapered or oval shape (e.g., like a football), in which the diameter of the bag varies along its length. It should thus be further understood that the diameter and length of the bag, or bags, may vary based on each embodiment. For example, the size of the patient (e.g., large adult, small adult, child, etc.) may require a different sized breathing bag. As would be understood by one skilled in the art, all of the figures and descriptions herein are subject to change in both size and relative location.

It should be noted that the system and processes of the figures are not exclusive. Other systems, processes, and menus may be derived in accordance with the principles of embodiments described herein to accomplish the same objectives. It is to be understood that the embodiments and variations shown and described herein are for illustration purposes only. Modifications to the current design may be implemented by those skilled in the art, without departing from the scope of the embodiments. As described herein, the various systems, subsystems, agents, managers, and processes can be implemented using hardware components, software components, and/or combinations thereof. No claim element herein is to be construed under the provisions of 35 U.S.C. 112(f) unless the element is expressly recited using the phrase “means for.”

Although the disclosure has been described with reference to exemplary embodiments, it is not limited thereto. Those skilled in the art will appreciate that numerous changes and modifications may be made to the embodiments described herein and that such changes and modifications may be made without departing from the true spirit of the disclosure. It is therefore intended that the appended claims be construed to cover all such equivalent variations as fall within the true spirit and scope of the disclosure.

1. An analgesia delivery system comprising:
a breathing bag comprising:
an outer shell that creates a hollow inner cavity, the outer shell having a first end and a second end;
wherein the first end comprises a first opening, and wherein the second end comprises a second opening and a check-valve; and
at least one tube having at least one aperture along a portion of the at least one tube;
wherein the at least one tube forms an air-tight seal with the first opening, traverses the hollow inner cavity, and is mechanically coupled to the check-valve, and
wherein the at least one aperture is between the first opening and the check-valve.

2. The analgesic delivery system of claim 1, wherein the at least one tube further comprises an inlet and an outlet;

3. The analgesic delivery system of claim 2, wherein the inlet is connected to at least one flowmeter.

4. The analgesic delivery system of claim 2, wherein the outlet is connected to at least one vacuum pump.

5. The analgesic delivery system of claim 2, wherein the inlet comprises an inlet coupling mechanism, and wherein the outlet comprises an outlet coupling mechanism.

6. The analgesic delivery system of claim 5, wherein the inlet coupling mechanism is configured to couple the at least one tube to a fresh gas supply, and wherein the outlet coupling mechanism is configured to couple the at least one tube to a vacuum system.

7. The analgesic delivery system of claim 1, wherein the outer shell is in a cuboid shape.

8. The analgesic delivery system of claim 1, wherein the outer shell is in an ellipsoid or spheroid shape.

9. The analgesic delivery system of claim 1, wherein the outer shell is collapsible.

10. The analgesic delivery system of claim 1, wherein the at least one aperture is selected from the group consisting of: slits, spiral cuts, geometric cut outs, and mesh tubing.

11. The analgesic delivery system of claim 1, wherein the check-valve comprises an inlet side, an outlet side, an air passage component, a tube attachment component, and a flapper component.

12. The analgesic delivery system of claim 11, wherein the air passage component comprises one or more openings configured to allow air passage.

13. The analgesic delivery system of claim 12, wherein the flapper component contacts the one or more openings, thereby restricting air passage.

14. The analgesic delivery system of claim 13, wherein the flapper component is configured to open when an air pressure on the outlet side is lower than an air pressure on the inlet side.

15. The analgesic delivery system of claim 11, wherein the tube attachment component is connected to the at least one tube to form an air tight seal.

16. The analgesic delivery system of claim 11, wherein the outlet side of the check-valve is coupled to a gas delivery tube.

17. The analgesic delivery system of claim 16, wherein the gas delivery tube is coupled to a patient delivery device.

18. The analgesic delivery system of claim 1, wherein the at least one tube and the breathing bag are fluidly coupled via the at least one aperture.

19. The analgesic delivery system of claim 1, wherein the at least one aperture comprises a plurality of apertures.

20. An analgesia delivery system comprising:
a breathing bag comprising:
an outer shell that creates a hollow inner cavity, the outer shell having a first end and a second end;
wherein the first end comprises a first opening, and wherein the second end comprises a second opening and a check-valve, and
wherein the check-valve comprises an inlet side, an outlet side, an air passage component, a tube attachment component, and a flapper component; and
at least one tube having at least one aperture along a portion of the at least one tube;
wherein the at least one tube forms an air-tight seal with the first opening, traverses the hollow inner cavity, and is mechanically coupled to the check-valve,
wherein the at least one aperture is located between the first opening and the check-valve,
wherein the outlet side of the check-valve is coupled to a gas delivery tube, and
wherein the gas delivery tube is coupled to a patient delivery device.
